(19) United States
(12) Patent Application Publication
(10) Pub. No.: US 2026/0217010 A1
(43) Pub. Date: Jul. 30, 2026

(54) METHOD AND APPARATUS FOR DISPENSING BARRIER FILM ON A MEDICAL DEVICE

(71) Applicant: Mitchell SKROSKI, Sr., Asheville, NC (US)

(72) Inventor: Mitchell SKROSKI, Sr., Asheville, NC (US)

(21) Appl. No.: 19/141,958

(22) PCT Filed: Feb. 16, 2024
(86) PCT No.: PCT/US2024/016192

§ 371 Date: Jun. 20, 2025

Related U.S. Application Data
(60) Provisional application No. 63/476,256, filed on Dec. 20, 2022.

(51) Int. Cl. B32B 37/00; A61B 90/00; A61L 31/16; B32B 38/00; B65H 19/10

(52) U.S. Cl. CPC B32B 37/0046; A61B 90/08; A61L 31/16; B32B 38/0004; B65H 19/10; A61L 2420/02; B32B 2535/00

(57) ABSTRACT

A film dispenser and method for dispensing a barrier film onto a medical device. The film dispenser includes a housing having a front wall, side walls, and a bottom wall; a rear wall pivotally connected to the housing; and a lid pivotally connected. An interior space is formed between the front wall, the side walls, and the bottom wall and enclosable by the rear wall. The front wall includes a slot for dispensing the barrier film, the front wall of the housing on a lower portion includes an indented portion such that the barrier film is able to be applied to the medical device. When the lid is in the open position, the barrier film is able to be dispensed from the interior space, and when the lid is in the closed position, the lid is configured to at least hold a portion of the barrier film.

### CROSS-REFERENCE

This application claims the benefit of provisional application 63/476256, filed Dec. 20, 2022, which is incorporated by reference.

The present disclosure relates to preventing and/or minimizing the exposure to hospital-acquired infections during use of a medical device. More specifically, the present disclosure relates to methods and apparatuses for dispensing a barrier film onto a medical device, such as a stethoscope, in which the transmission of infectious materials and/or contaminants is minimized or prevented, for example, by applying the barrier film onto the medical device.

### BACKGROUND

Hospital-acquired infections (HAIs) pose a great threat to the health and life of patients across the healthcare fields, including acute care hospitals, skilled nursing facilities and outpatient environments such as primary care providers (PCP) and specialty hospital clinics, surgery centers and physician offices. HAIs are generally considered as being acquired from acute & post-acute settings. For example, the HAIs may be acquired from the use of medical devices, such as stethoscopes. Healthcare professionals (doctors, nurses, etc.) are required to clean stethoscopes between patients, for example, for 60 seconds, and more often than not, even if the stethoscope are cleaned, the infectious material and/or contaminants, such as, bacteria and infectious pathogens may still be present. Transmission of infectious materials and/or contaminants typically occurs in one out of every 32 patient encounters. The Centers for Disease Control and Prevention recognize that HAIs are one of the major problems in US hospitals, thus, a significant need exists for protection from one of the major contributors to the risk of HAIs to patients. While products are available for allegedly delivering effective barrier protection for the patient, such products have high costs and require extensive maintenance, such as, having certified technicians calibrate each device monthly.

### SUMMARY

In order to prevent or minimize the exposure of HAIs to patients from medical devices, such as from a stethoscope, the present disclosure is directed to methods and apparatuses for dispensing a barrier film onto the medical device, in which the transmission of infectious materials and/or contaminants is minimized or prevented, for example, by applying the barrier film onto the medical device to reduce or prevent transmission from the medical device to the patient.

In an embodiment, a film dispenser for dispensing a barrier film from a roll of film onto a medical device is provided. The film dispenser includes a housing having a front wall, side walls, and a bottom wall; a rear wall pivotally connected to the housing; and a lid pivotally connected to the front wall of the housing. The housing includes an interior space formed between the front wall, the side walls, and the bottom wall and enclosable by the rear wall, the interior space being configured to receive the roll of film that is positionable along the side walls. The front wall includes a slot for dispensing the barrier film from the roll of film in the interior space along the front wall of the housing, the front wall of the housing on a lower portion thereof including an indented portion configured to engage the medical device such that the barrier film is able to be applied to the medical device, and the lid is configured such that when the lid is in the open position, the barrier film is able to be dispensed from the interior space, and when the lid is in the closed position, the lid is configured to at least hold a portion of the barrier film.

In another embodiment, a method of replacing a roll of film from a film dispenser for a medical device, the film dispenser comprising a housing having a front wall, side walls, and a bottom wall; and a rear wall pivotally connected to the housing, wherein the housing includes an interior space formed between the front wall, the side walls, the bottom wall, and the rear wall is provided. The method includes releasing the housing from the rear wall; pivotally opening the housing from the rear wall to access the interior space; removing the roll of film from the interior space from the side walls; inserting a new roll of film into the interior space along the side walls; feeding barrier film from the roll of film through a slot in the front wall for dispensing the barrier film from the roll of film along the front wall of the housing; and pivotally closing the housing against the rear wall, wherein during the closing of the housing, the housing is connected to the rear wall.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIGS. 1A, 1B, 1C illustrate an example film dispenser, according to an embodiment.

FIG. 2 illustrates a rear view of an example film dispenser, according to an embodiment.

FIG. 3 illustrates an example container or cartridge having a roll of film, according to an embodiment.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

As discussed above, HAIs pose a great threat to the health and life of patients across the healthcare fields, in which the HAIs may be acquired by the patient from the use of medical devices, such as stethoscopes. The source of the HAIs may be from improper cleaning and/or due to remnants or portions of the infectious material or contaminants, such as, bacteria and infectious pathogens, still being present on the medical device. While products are available for allegedly delivering effective barrier protection for the patient, such products have high costs and/or require extensive maintenance, such as, having certified technicians calibrate each device monthly. Moreover, some of the products that are available still require some contact by the healthcare professional when applying or replacing the barrier protection, which reduces the effectiveness of the barrier protection, since contaminants and/or infectious material may be transferred from the healthcare professional to the barrier film, e.g., when the healthcare professional touches the barrier protection, which may transfer contaminants and/or infectious material to the barrier film.

Embodiments as discussed herein are directed to overcoming one or more of the deficiencies of the prior products that provide barrier protection. In some embodiments, the present disclosure is directed to methods and apparatuses for dispensing a barrier film onto a medical device, in which the transmission of infectious materials and/or contaminants to the patient is minimized or prevented. That is, in order to prevent or minimize the exposure of HAIs to patients from medical devices, such as, from a stethoscope, the methods and apparatuses as discussed herein are directed to allowing a healthcare professional to not only apply the barrier film to a medical device with low or minimal transmittance of contaminants or infectious material from the healthcare professional to the medical device, but also provide the barrier film to minimize or prevent transmittance of the contaminants or infectious material from the medical device to the patient.

For example, in some embodiments, the apparatus may include a film dispenser for dispensing a barrier film from a roll of film, the film dispenser having a rear wall that is mountable onto a wall or other supporting structure. As such, the healthcare professional may be able to dispense a portion of a thin plastic membrane of a barrier film having at least one surface adherable to the stethoscope from a roll of material contained in an interior space of the film dispenser, in which there is minimal or no contact by the healthcare professional when applying the barrier film to the stethoscope, such that there is no or small amounts of transmission of bacterial or viral contaminants between the healthcare professional and the patient, e.g., 100% clean contact with the patient. The film dispenser may also allow the application of the barrier film to the stethoscope, e.g., the drumhead or diaphragm, such that the transmission of contaminants or infectious materials, which may be a result of poor hygiene of the stethoscope, from the stethoscope to the patient is minimized or prevented. The film dispenser may be designed or otherwise configured such that the film dispenser may be located in a plurality of locations, such as, exam rooms, acute care patient rooms, in clinics, such as, occupational health clinics, educational clinics and settings, surgery centers, EMT vehicles, or the like in which medical devices, such as, stethoscopes, may be used. In some embodiments, the film dispenser may be portable for individual use, for example, portable in a lab coat, pocket, or purse. In some embodiments, the barrier film may have between 0.5 and 10 mil thickness, and in some embodiments 1 to 3 mil thicknesses, and even further, having a 1 mil thickness.

The barrier film may be formed from polyethylene, polyolefin, bioplastics, medical grade tape, soft polyvinyl chloride (PVC), such as, “cling film”, polyamide, polyethylene terephthalate, ethylene-vinyl alcohol, mylar, VMPET, BOPET, METPET, polyvinylidene chloride, or other similar films and may further contain antimicrobial properties by including one or more of silver, copper, titanium, or any other antimicrobial ingredient, property, surface, agent, or properties that may be used to reduce the spread of infectious pathogens or bacteria. It is appreciated that the barrier film may include a single film layer or may include multiple layers of any of the combinations discussed above.

Various embodiments and examples are discussed further below.

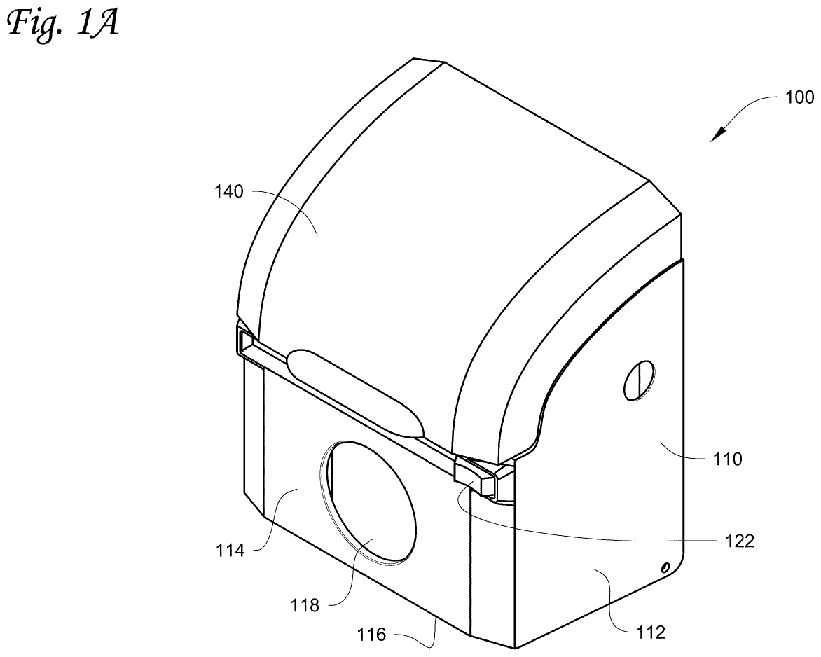

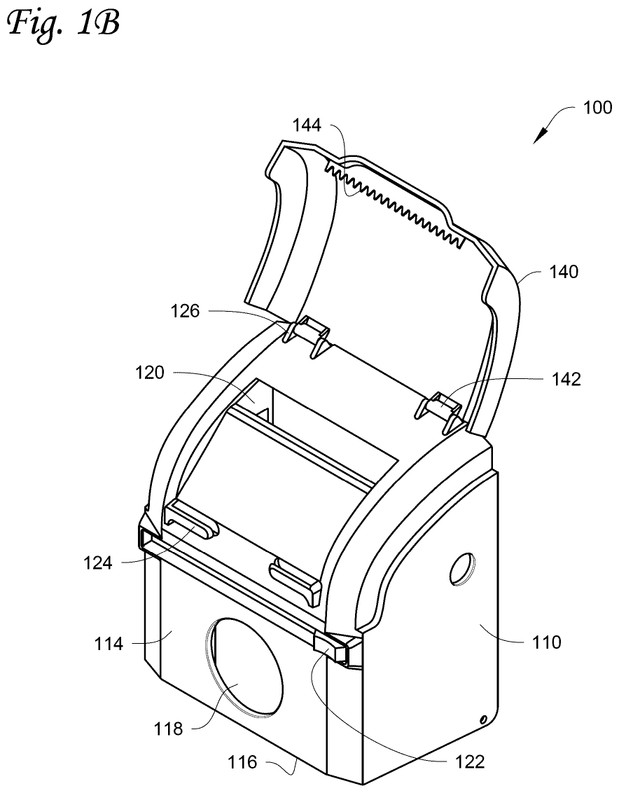

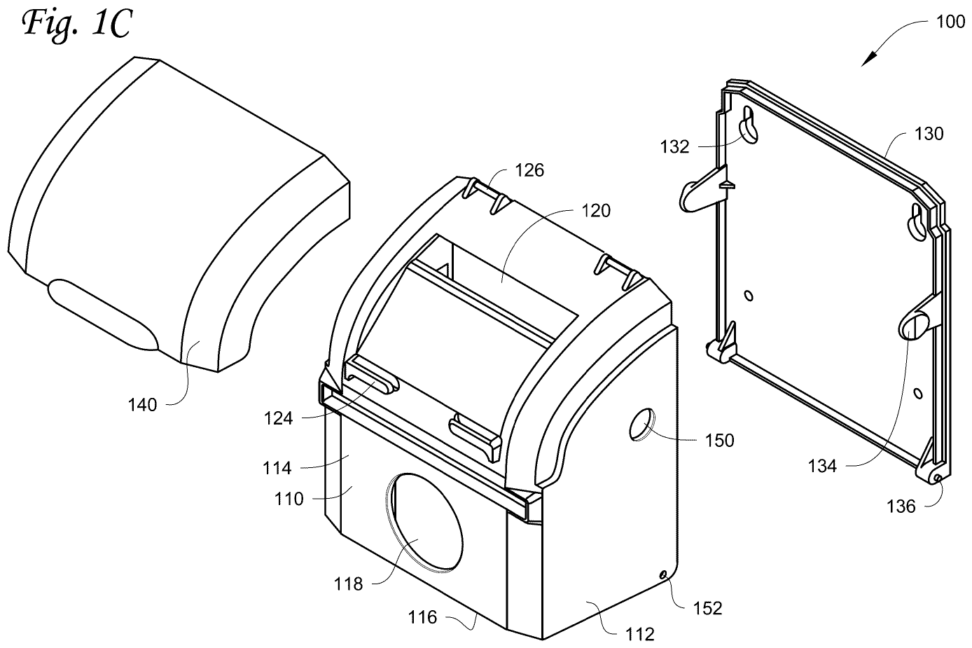

FIGS. 1A, 1B, 1C illustrate a film dispenser 100 according to an embodiment. The film dispenser 100 includes a housing 110, a rear wall 130, and a lid 140. The film dispenser 100 may be designed or otherwise configured to allow a healthcare professional, such as, a doctor, nurse, physicians assistant, emergency medical technician, or the like, to apply a barrier film from a roll of film onto a medical device, such as, a stethoscope, with little to no contact of the barrier film. As such, the barrier film may be applied to the medical device such that that there is little to no transmission of infectious material or contaminants from the healthcare professional to the barrier film or medical device or from the medical device to the patient(s), in which the infectious material or contaminant, such as, bacteria or viral contaminants may be a result of poor hygiene and/or any contaminants or infectious material being present on the medical device.

In some embodiments, the housing 110 may include side walls 112, e.g., right and left side walls 112, a front wall 114, and a bottom wall 116. The side walls 112, the front wall 114, and bottom wall 116 may be attached to each other via fasteners, such as, screws, bolt, rivets, or the like, or may be integrally formed as a unitary piece, e.g., molded as a single piece. The side walls 112, the front wall 114, and bottom wall 116 enclose and/or form an interior space for receiving a roll of film for dispensing a barrier film.

The front wall 114 may include an indented portion 118, a slot 120, a barrier film cutting device 122, a guiding portion 124, and pivot fastening portions 126. The front wall 114 may be a curved wall that is joined along its side edges to the side walls 112 and along a bottom edge to the bottom wall 116. In some embodiments, a ledge interface may be formed between the front wall 114 and the side walls 112. The slot 120 may be provided or positioned near or below a top edge of the front wall 114. The slot 120 may be designed or otherwise configured to allow for the dispensing of the barrier film from the roll of film in the interior space through the front wall 114 and along a front surface of the front wall 114. The guiding portions 124 may be provided below the slot 120 at or near the middle of the front wall 114 that are designed or configured to guide the barrier film along the front wall of the housing, e.g., to keep the barrier film near or adjacent the front surface, and/or remove the barrier film from the lid 140 when lifted. That is, in some embodiments, the guiding portions 124 may be designed or otherwise configured to maintain the barrier film along the front wall 114, e.g., provide a pathway for the medical device, e.g., stethoscope, to stay in contact with the front wall 114 until the medical device reaches the indented portion 118. The indented portion 118 may be provided near or above the bottom edge of the front wall 114 and designed or otherwise configured to engage a medical device. In some embodiments, the indented portion 118 may be a cavity having a lip formed in the front wall 114, in which the cavity has a geometric shape, such as, circular, rectangular, triangular, or the like. As such, the indented portion 118 may be designed or otherwise configured to engage at least a portion of the medical device, such as, a distal end of a stethoscope, such that the portion of the medical device may be rotated within the cavity and at least partially beneath the lip for applying the barrier film to the medical device, e.g., to the front surface and at least a part of the back surface of the medical device. In some embodiments, the front wall may further include the barrier film cutting device 122 which may be provided between the indented portion 118 and the slot 120. The barrier film cutting device 122 may include an edged device, such as, a razor, knife, or the like, that may be slid along a track for cutting or separating the barrier film from the roll of film. Moreover, in some embodiments, the front wall 114 may further include the pivot fastening portions 126 provided near or at the top edge. The pivot fastening portions 126 may be a rod, bar, or other clip receiving portion for receiving pivoting clips or clamps from the lid 140, as further discussed below.

The side walls 112 may be designed or otherwise configured to have a top edge that has the same or similar shape of at least a portion of the front wall 114, e.g., curved. As indicated above, the front wall 114 and the side walls may form the ledge at an interface thereof. The side walls 112 may include corresponding portions 150 that correspond to engagement portions 134 (discussed below) for connecting or disconnecting the housing 110 from the rear wall 130. In some embodiments, the corresponding portions 150 may be openings for receiving the flexible tabs, magnets, hooks or loops, clips, or the like. The side walls 112 may further include pivot holes 152 for receiving pivoting connections 136 (discussed below) for pivoting or rotating the housing 110 with respect to the rear wall 130. In some embodiments, the side walls 112 may include along an inner surface thereof film receiving portions for receiving the roll of film. The roll of film may be provided on a spool and/or provided in a container or cartridge for minimizing contact with the barrier film, e.g., to reduce or prevent the transmittance of the infectious material or contaminants, such as, bacteria, infectious pathogens, or the like, from the healthcare worker to the barrier film. In some embodiments, the roll of film may include a spring loaded spool and/or ends for being received in the receiving portions of the side walls 112.

The lid 140 may include pivot clamping portions 142 and fixing portions 144. The lid 140 may be designed or otherwise configured to have the same or similar shape as the front wall 114, e.g., curved. In some embodiments, the lid 140 may have walls at side edges thereof for engaging or being positioned against the ledge interface between the front wall 114 and the side walls 112. As discussed above, the lid 140 may be pivotally connected to the front wall 114 by having the pivot clamping portions 142 connected or coupled to the pivot fastening portions 126 on the front wall 114. The pivot clamping portions 142 may be clips or clamps, e.g., C-shaped clips, that are elastically deformable such that the clips or clamps may be connected, e.g., a snap-fit connection, to the pivot fastening portions 126 and designed or otherwise configured for rotation around the pivot fastening portions 126. The fixing portions 144 may be protrusions or teeth for contacting the barrier film that are designed or otherwise configured for holding or engaging the barrier film to prevent or minimize the movement of the barrier film when applying the barrier film to the medical device and/or when cutting or separating the barrier film from the roll of film, e.g., by pressing the barrier film against the front wall 114. It is appreciated that since at least one side of the barrier film is adherable to a surface, e.g., static adherence or pressure sensitive adhesive, the contact surface(s) of the fixing portions 144 may be minimized to reduce the adherence of the barrier film to the fixing portions 144. In some embodiments, a row of teeth may be provided such that the fixing portions 144 only have point contacts with the barrier film. In other embodiments, a plurality of protrusions may be provided, such as, between two and ten protrusions, such that the total contact surface may be reduced. In some embodiments, the guiding portions 124 of the front wall 114 may be designed or otherwise configured to remove the barrier film from the fixing portions 144 when the lid 140 is opened.

As illustrated in FIG. 1C, the film dispenser 100 further includes the rear wall 130. The rear wall 130 includes fixing holes 132, engagement portions 134, and pivoting connections 136. The rear wall 130 has a shape corresponding to the housing 110 such that the rear wall 130 is designed or otherwise configured to enclose the interior space formed in the housing 110, when the rear wall 130 is attached or connected thereto. The fixing holes 132 may be designed or configured to receive a fastener, such as, nail, screw, bolt, or the like, for securing the film dispenser 100 to a structural support, such as, a wall, board, stand, or the like. The engagement portions 134 may be provided along side edges of the rear wall 130 and may extend towards the housing 110. In some embodiments, the engagement portions may be designed or otherwise configured to allow engagement and disengagement between the housing 110 and the rear wall 130. In some embodiments, the engagement portions 134 may be flexible tabs that are elastically deformable such that the engagement portions 134 may form a snap-fit connection with the housing 110. As such, the housing 110 may be disconnected from the rear wall 130 by depressing the engagement portions 134 and connected to the rear wall 130 by pressing the housing 110 against the rear wall 130 such that the engagement portions 134 are elastically deformed and restored when the engagement portions 134 are engaged with the corresponding portions 150 on the housing 110, e.g., the engagement portions 134 may have a cantilever snap-fit design. While an embodiment of the engagement portion 134 has been discussed herein, such disclosure is not intended to be limiting. Rather, it is understood that the engagement portion 134 may have various designs that allow the connection and disconnection of the housing 110 from the rear wall, e.g., annular snap-fit design, torsional snap-fit design, hook and loop fastener, clips, magnets, or the like. The pivoting connections 136 may include pins or protrusions that are designed or otherwise configured to engage the pivot holes 152 in the housing 110. The pivoting connections 136 may be provided at or near the bottom edge of the rear wall 130 such that when the housing 110 is connected to the rear wall 130, e.g., via the pivot holes 152 in the side walls 112 or hinged connection to the bottom wall 116, the housing 110 is pivotally connected to allow the housing 110 to be rotated, e.g., rotated away from the rear wall 130, e.g., in a downward 90 degree rotation, to open and/or allow access to the interior space and rotated towards the rear wall 130 for enclosing the interior space.

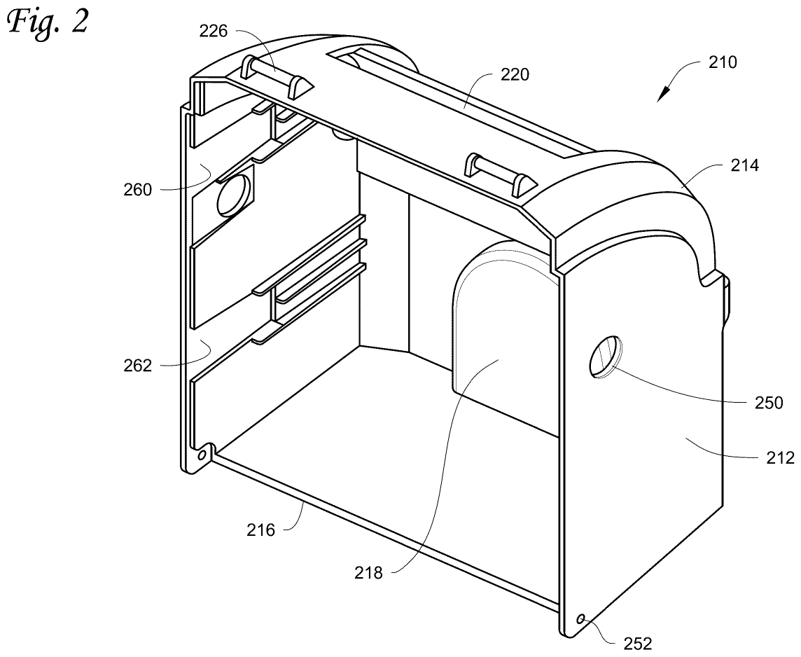

FIG. 2 illustrates a rear-view of an embodiment of a housing 210 for a film dispenser according to another embodiment. The film dispenser may include the same or similar features of the film dispenser 100 with respect to FIG. 1, as discussed above, which are not discussed further below for sake of clarity.

In some embodiments, the housing 210 may include side walls 212, e.g., right and left side walls 212, a front wall 214, and a bottom wall 216. The side walls 212, the front wall 214, and bottom wall 216 may be attached to each other via fasteners, such as, screws, bolt, rivets, or the like, or may be integrally formed as a unitary piece, e.g., molded as a single piece. The side walls 212, the front wall 214, and bottom wall 216 enclose and/or form an interior space for receiving a roll of film for dispensing a barrier film.

The front wall 214 may include an indented portion 218, a slot 220, and pivot fastening portions 226. The front wall 214 may be a curved wall that is joined along its side edges to the side walls 212 and along a bottom edge to the bottom wall 216. The slot 220 may be provided or positioned near or below a top edge of the front wall 214. The slot 220 may be designed or otherwise configured to allow for the dispensing of the barrier film from the roll of film in the interior space through the front wall 214 and along a front surface of the front wall 214. The front wall 214 may further include the pivot fastening portions 226 provided near or at the top edge.

The pivot fastening portions 226 may be a rod, bar, or other clip receiving portion for receiving pivoting clips or clamps from the lid, e.g., 140.

The side walls 212 may be designed or otherwise configured to have a top edge that has the same or similar shape of at least a portion of the front wall 214, e.g., curved. The side walls 212 may include corresponding portions 250 that correspond to engagement portions, e.g., 134, for connecting or disconnecting the housing 210 from the rear wall, e.g., 130. In some embodiments, the corresponding portions 250 may be openings for receiving the flexible tabs, magnets, hooks or loops, clips, or the like. The side walls 212 may further include pivot holes 252 for receiving pivoting connections, e.g., 136, for pivoting or rotating the housing 210 with respect to the rear wall, e.g., 130.

As seen in the rear view of FIG. 2, the side walls 212 may further include along an inner surface a pair of notched grooves 260 in the side walls 212. The pair of notched grooves 260 may include a channel for receiving engagement ends of a roll of film or cartridge or container having a roll of film, e.g., designed or otherwise configured for insertion and/or positioning of the roll of film into the notched grooves. The pair of notched grooves 260 may further include an abutment surface, e.g., raised rib or portion of the side wall 212, for positioning the roll of film or cartridge or container within the interior space. In some embodiments, the side walls 212 may further include a second pair of notched grooves 262, having the same or similar features as the pair of notched grooves 260, for receiving a second (or spare) roll of film or cartridge or container of film. As such, the housing 210 may include a second roll of film that may be used to replace the roll of film in the pair of notched grooves 260 when the roll of film is empty.

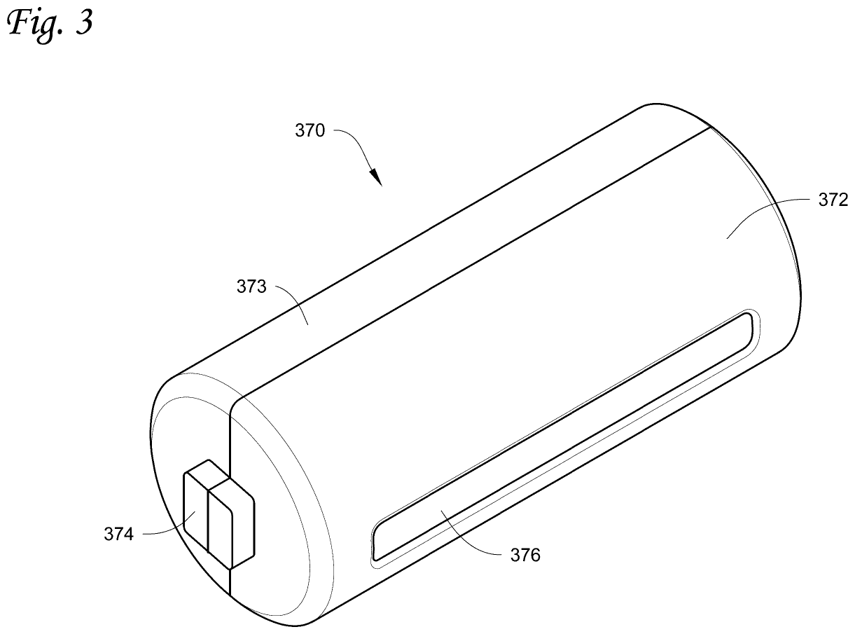

FIG. 3 illustrates an example embodiment of a cartridge or container for a roll of film 370. The cartridge or container for the roll of film 370 may be inserted into the film dispenser 100 of FIG. 1 or 200 of FIG. 2.

The cartridge or container for the roll of film 370 may include housing parts 372, 373, such as upper and lower cylindrical housing parts, that may include a snap-fit connection for connecting or disconnecting the same. As such, the housing parts 372, 373 may include a roll of film inserted therein, such that the housing parts 373, 373 may be connected/disconnected to enclose the roll of film. At least one of the housing parts 373, 373 may include a slot opening 376 for dispensing the barrier film from the roll of film, such that the barrier film may be dispensed through the slot, e.g., 120, of the front wall, e.g., 114. The cartridge or container for the roll of film 370 may further include engagement ends 374 that correspond to a pair of notched grooves, e.g., 260, in side walls, e.g., 212, of a housing, e.g., 210. The engagement ends 374 may have a geometric profile, such as, rectangular, circular, triangular, or the like, for being received in the pair of notched grooves.

Operation of a film dispenser 100 of FIG. 1 (or 200 of FIG. 2) having a roll of film, such as, cartridge or container 370 of FIG. 3 is discussed further below.

Specifically, the roll of film 370 may be provided in the interior space of the housing 110 of the film dispenser 100, e.g., along the pair of notched grooves, e.g., 260 of FIG. 2, in which the interior space is enclosed by the rear wall 130. The barrier film may be provided or fed from the roll of film through the slot 120 of the front wall 114 of the film dispenser 100. In some embodiments, the barrier film may be provided along a front surface of the front wall 114. When the film dispenser 100 is not in use, the lid 140 may be closed to cover at least a portion of the front wall 114 which may include the barrier film to prevent or minimize the contamination of the barrier film from bacteria or other viral contaminants, e.g., atmospheric biologics.

When the film dispenser 100 is in use, the healthcare provider may apply the barrier film to a medical device, such as, a distal end of a stethoscope, by opening the lid 140, e.g., pivotally rotating the lid 140 away from the front wall 114, to expose the barrier film on a portion of the front wall 114. The healthcare provider may then dispense the barrier film from the roll of film and guide the barrier film downward to the indented portion 118 of the front wall 114 and subsequently close the lid 140 to hold at least a portion of the barrier film, e.g., via fixing portions 144 which may press the barrier film against the front wall 114. It is appreciated that the indented portion 118 may be designed or otherwise configured as a stopper such that a proper length of barrier film is dispensed for application to the medical device, e.g., 3-5 inches or 7.62 cm to 12.7 cm. With the lid 140 in the closed position, the barrier film cutting device 122 may be used to cut or separate the barrier film from the remainder of the roll of film, e.g., by evenly cutting the barrier film along the track, such that the barrier film is applied to the medical device. The healthcare provider may then rotate the medical device, e.g., drumhead or diaphragm of the stethoscope, inside the indented portion 118 to apply the barrier film on at least the front surface of the medical device and at least a portion on a back surface thereof.

In some embodiments, the front wall 114 may include the guiding portions 124, such that when the lid 140 is opened, the guiding portions 124 may be designed or otherwise configured to hold or remove the barrier film from the lid 140, e.g., portions of the lid that are adhered to the barrier film, and/or for guiding the barrier film along the front surface of the front wall 114.

The film dispenser 100 (or 200) may be further designed or otherwise configured to allow the replacement of the roll of film with minimal or no contact of the barrier film such that minimal or no contaminants or infectious materials are transferred from the healthcare professional to the barrier film. For example, in some embodiments, in order to replace the roll of film, the housing 110 may be released from the rear wall 130 such that the housing 110 may be pivotally opened to access the interior space, e.g., pivotally rotated 90 degrees away from the rear wall 130. In some embodiments, the housing 110 may be released from the rear wall 130 by depressing the engagement portions 134, e.g., flexible tabs, on the rear wall 130 to disengage the engagement portions 134 from the corresponding portions 150, e.g., the openings, of the housing 110. As such, the housing 110 is releasable and separable from the rear wall 130.

After the housing 110 is pivotally rotated away from the rear wall 130, the roll of film may be removed from the interior space, e.g., from the pair of notched grooves, e.g., 260, provided in the side walls, e.g., 112 or 212. A new roll of film may then be inserted into the interior space, e.g., along the pair of notched grooves of the side walls. The barrier film may then be dispensed from the new roll of film and fed through the slot 120 of the front wall 114 for dispensing the barrier film from the roll of film along the front wall of the housing 110. The housing 110 may then be pivotally rotated to close the interior space by connecting the housing 110 to the rear wall 130. In some embodiments, the closing of the housing 110 against the rear wall 130 may include elastically deforming the engagement portions 134, e.g., flexible tabs, such that the engagement portions 134 are restored to their original positions when engaged with the corresponding portions 150 on the housing 110 when the housing 110 is pressed against the rear wall 130, e.g., the flexible tabs form a snap-fit connection with the holes in the housing 110.

As such, the film dispenser, as discussed herein, may be designed or otherwise configured to allow a healthcare professional, such as, a doctor, nurse, physicians assistant, emergency medical technician, or the like, to apply a barrier film from a roll of film onto a medical device, such as, a stethoscope, with little to no contact of the barrier film (or roll of film), such that there is little to no transmission of infectious material or contaminants from the healthcare professional to the barrier film. Moreover, since the barrier film is applied to the medical device there is little to no transmission of infectious material or contaminants from the medical device to the patient(s), in which the infectious material or contaminant, such as, bacteria or viral contaminants may be a result of poor hygiene and/or any contaminants or infectious material being present on the medical device. Accordingly, a film dispenser is provided that minimizes or prevents the transmission of contaminants and/or infectious materials to a patient that has a low cost of manufacture and does not require extensive maintenance.

### ASPECTS

It is appreciated that any one of the aspects may be combined with each other.

Aspect 1: A film dispenser for dispensing a barrier film from a roll of film onto a medical device, the film dispenser comprising: a housing having a front wall, side walls, and a bottom wall; a rear wall pivotally connected to the housing; and a lid pivotally connected to the front wall of the housing, wherein the housing includes an interior space formed between the front wall, the side walls, and the bottom wall and enclosable by the rear wall, the interior space being configured to receive the roll of film that is positionable along the side walls, wherein the front wall includes a slot for dispensing the barrier film from the roll of film in the interior space along the front wall of the housing, the front wall of the housing on a lower portion thereof including an indented portion configured to engage the medical device such that the barrier film is able to be applied to the medical device, and wherein the lid is configured such that when the lid is in the open position, the barrier film is able to be dispensed from the interior space, and when the lid is in the closed position, the lid is configured to at least hold a portion of the barrier film.

Aspect 2. The film dispenser according to Aspect 1, wherein the roll of film is positionable along the side walls along a pair of notched grooves provided on the side walls.

Aspect 3. The film dispenser of Aspect 2, wherein the roll of film is provided inside a cartridge, the cartridge having engagement ends corresponding to the pair of notched grooves provided along the side walls of the housing for insertion into the interior space.

Aspect 4. The film dispenser of any of Aspects 1-2, wherein the rear wall is pivotally connected to bottom portions of the side walls.

Aspect 5. The film dispenser of any of Aspects 1-4, wherein the rear wall includes engagement portions for engaging or disengaging corresponding portions on the housing for connecting or disconnecting the housing from the rear wall.

Aspect 6. The film dispenser of Aspect 5, wherein the engagement portions are flexible tabs, such that the housing is disconnected from the rear wall when the flexible tabs are depressed.

Aspect 7. The film dispenser of any of Aspects 1-6, wherein in the interior space, the side walls further include a second pair of notched grooves for receiving a second film in the interior space thereof.

Aspect 8. The film dispenser of any of Aspects 1-7, wherein the front wall, the side walls, and the bottom wall are integrally formed.

Aspect 9. The film dispenser of any of Aspects 1-8, wherein the lid is pivotally connected to a top portion of the front wall.

Aspect 10. The film dispenser of any of Aspects 1-9, wherein the lid comprises fixing portions for holding the film.

Aspect 11. The film dispenser of Aspect 10, wherein the fixing portions are protrusions or teeth for contacting the film.

Aspect 12. The film dispenser of any of Aspects 1-11, wherein the front wall further includes a guiding portion for guiding the film along the front wall.

Aspect 13. The film dispenser of any of Aspects 1-12, wherein the front wall includes a cutting portion for cutting the film.

Aspect 14. A method of replacing a roll of film from a film dispenser for a medical device, the film dispenser comprising a housing having a front wall, side walls, and a bottom wall; and a rear wall pivotally connected to the housing, wherein the housing includes an interior space formed between the front wall, the side walls, and the bottom wall and enclosable by the rear wall, the method comprising: releasing the housing from the rear wall; pivotally opening the housing from the rear wall to access the interior space; removing the roll of film from the interior space from the side walls; inserting a new roll of film into the interior space along the side walls; feeding barrier film from the roll of film through a slot in the front wall for dispensing the barrier film from the roll of film along the front wall of the housing; and pivotally closing the housing against the rear wall, wherein during the closing of the housing, the housing is connected to the rear wall.

Aspect 15. The method of Aspect 14, wherein the releasing the housing from the rear wall includes depressing flexible tabs on the rear wall to disengage the flexible tabs from corresponding portions on the housing.

Aspect 16. The method of any of Aspects 14-15, wherein the closing of the housing against the rear wall includes elastically deforming flexible tabs on the rear wall, such that the flexible tabs are restored to original positions when engaged with the corresponding portions on the housing.

Aspect 17. The method of any of Aspects 14-16, wherein the roll of film is provided inside a cartridge, the cartridge having engagement ends corresponding to a pair of notched grooves provided along the side walls of the housing for insertion into the interior space.

Aspect 18. The method of any of Aspects 14-16, wherein in the interior space, the side walls further include a second pair of notched grooves for receiving a second roll of film in the interior space thereof.

Aspect 19. The method of Aspect 18, wherein the inserting the new roll of film includes removing the second roll of film from the second pair of notched grooves and inserting the second film as the new roll of film.

Aspect 20. The method of any of Aspects 18-19, wherein the second roll of film is provided in a second cartridge, the second cartridge having engagement ends corresponding to both the pair of notched grooves and the second pair of notched grooves provided along the side walls of the housing for insertion into the interior space.

The foregoing description is presented to enable one of ordinary skill in the art to make and use the disclosed embodiments and modifications thereof, and is provided in the context of a patent application and its requirements. Various modifications to the disclosed embodiments and the principles and features described herein will be readily apparent to those of ordinary skill in the art. Thus, the present disclosure is not intended to limit the invention to the embodiments shown; rather, the invention is to be accorded the widest scope consistent with the principles and features described herein.

What is claimed is:

1. A film dispenser for dispensing a barrier film from a roll of film onto a medical device, the film dispenser comprising:
a housing having a front wall, side walls, and a bottom wall;
a rear wall pivotally connected to the housing;
and a lid pivotally connected to the front wall of the housing,
wherein the housing includes an interior space formed between the front wall, the side walls, and the bottom wall and enclosable by the rear wall, the interior space being configured to receive the roll of film that is positionable along the side walls,
wherein the front wall includes a slot for dispensing the barrier film from the roll of film in the interior space along the front wall of the housing, the front wall of the housing on a lower portion thereof including an indented portion configured to engage the medical device such that the barrier film is able to be applied to the medical device, and
wherein the lid is configured such that when the lid is in the open position, the barrier film is able to be dispensed from the interior space, and when the lid is in the closed position, the lid is configured to at least hold a portion of the barrier film.

2. The film dispenser according to claim 1, wherein the roll of film is positionable along the side walls along a pair of notched grooves provided on the side walls.

3. The film dispenser of claim 2, wherein the roll of film is provided inside a cartridge, the cartridge having engagement ends corresponding to the pair of notched grooves provided along the side walls of the housing for insertion into the interior space.

4. The film dispenser of claim 1, wherein the rear wall is pivotally connected to bottom portions of the side walls.

5. The film dispenser of claim 1, wherein the rear wall includes engagement portions for engaging or disengaging corresponding portions on the housing for connecting or disconnecting the housing from the rear wall.

6. The film dispenser of claim 5, wherein the engagement portions are flexible tabs, such that the housing is disconnected from the rear wall when the flexible tabs are depressed.

7. The film dispenser of claim 1, wherein in the interior space, the side walls further include a second pair of notched grooves for receiving a second film in the interior space thereof.

8. The film dispenser of claim 1, wherein the front wall, the side walls, and the bottom wall are integrally formed.

9. The film dispenser of claim 1, wherein the lid is pivotally connected to a top portion of the front wall.

10. The film dispenser of claim 1, wherein the lid comprises fixing portions for holding the barrier film.

11. The film dispenser of claim 10, wherein the fixing portions are protrusions or teeth for contacting the barrier film.

12. The film dispenser of claim 1, wherein the front wall further includes a guiding portion for guiding the film along the front wall.

13. The film dispenser of claim 1, wherein the front wall includes a cutting portion for cutting the film.

14. A method of replacing a roll of film from a film dispenser for a medical device, the film dispenser comprising a housing having a front wall, side walls, and a bottom wall; and a rear wall pivotally connected to the housing, wherein the housing includes an interior space formed between the front wall, the side walls, and the bottom wall and enclosable by the rear wall, the method comprising:
releasing the housing from the rear wall;
pivotally opening the housing from the rear wall to access the interior space;
removing the roll of film from the interior space from the side walls;
inserting a new roll of film into the interior space along the side walls;
feeding barrier film from the roll of film through a slot in the front wall for dispensing the barrier film from the roll of film along the front wall of the housing; and
pivotally closing the housing against the rear wall, wherein during the closing of the housing, the housing is connected to the rear wall.

15. The method of claim 14, wherein the releasing the housing from the rear wall includes depressing flexible tabs on the rear wall to disengage the flexible tabs from corresponding portions on the housing.

16. The method of claim 14, wherein the closing of the housing against the rear wall includes elastically deforming flexible tabs on the rear wall, such that the flexible tabs are restored to original positions when engaged with the corresponding portions on the housing.

17. The method of claim 15, wherein the roll of film is provided inside a cartridge, the cartridge having engagement ends corresponding to a pair of notched grooves provided along the side walls of the housing for insertion into the interior space.

18. The method of claim 15, wherein in the interior space, the side walls further include a second pair of notched grooves for receiving a second roll of film in the interior space thereof.

19. The method of claim 18, wherein the inserting the new roll of film includes removing the second roll of film from the second pair of notched grooves and inserting the second film as the new roll of film.

20. The method of claim 18, wherein the second roll of film is provided in a second cartridge, the second cartridge having engagement ends corresponding to both the pair of notched grooves and the second pair of notched grooves provided along the side walls of the housing for insertion into the interior space.
